(19) United States
(12) Patent Application Publication
(10) Pub. No.: US 2023/0398355 A1
(43) Pub. Date: Dec. 14, 2023

(54) SYSTEMS AND METHODS FOR TREATING GASTROINTESTINAL DYSMOTILITY

(71) Applicants: Duke University, Durham, NC (US); Flinders Medical Center, Adelaide (AU)

(72) Inventors: Warren Grill, Durham, NC (US); Bradley Barth, Durham, NC (US); Nick Spencer, Adelaide (AU)

(21) Appl. No.: 18/250,090

(22) PCT Filed: Oct. 21, 2021
(86) PCT No.: PCT/US2021/055997

§ 371 Date: Apr. 21, 2023

Related U.S. Application Data
(60) Provisional application No. 63/094,991, filed on Oct. 22, 2020.

(51) Int. Cl. A61N 1/36

(52) U.S. Cl. CPC A61N 1/3606; A61N 1/36178; A61N 1/36171; A61N 1/36053

(57) ABSTRACT

The present disclosure provides systems and methods relating to the treatment of gastrointestinal dysmotility. In particular, the present disclosure provides systems and methods for delivering temporal patterns of electrical stimulation with respect to a refractory period to either suppress (e.g., treat hypermotility) or stimulate (e.g., treat hypomotility) contractions and motility in the gastrointestinal tract of a subject.

### RELATED APPLICATIONS

This application claims priority to and the benefit of U.S. Provisional Patent Application No. 63/094,991 filed Oct. 22, 2020, which is incorporated herein by reference in its entirety for all purposes.

### GOVERNMENT FUNDING

This invention was made with Government support under Federal Grant No. R01 DK119795 awarded by the National Institutes of Health National Institute of Diabetes & Digestive & Kidney Diseases (NIH/NIDDK). The Federal Government has certain rights to the invention.

### FIELD

The present disclosure provides systems and methods relating to the treatment of gastrointestinal dysmotility. In particular, the present disclosure provides systems and methods for delivering temporal patterns of electrical stimulation with respect to a refractory period to either suppress (e.g., treat hypermotility) or stimulate (e.g., treat hypomotility) contractions and motility in the gastrointestinal tract of a subject.

### BACKGROUND

Coordination of colonic motility relies on neurogenic motor patterns regulated by the enteric nervous system (ENS). The colonic motor complex (CMC) is one such motor pattern and has been reported in many species, including humans. The CMC is defined as “neurogenic repetitive peaks of pressure and/or electrical activity which can be migrating or nonmigrating in either the anterograde or retrograde directions.” Typically, CMCs are measured by force transducers or intraluminal pressure sensors, and the electrical corollary, the myoelectric complex (MC), is measured by intracellular or extracellular electrodes. MCs are typically associated with muscle action potentials and underlie the electrical component of the CMC contraction. Although each CMC contraction may not necessarily lead to propulsion, they catalyze self-sustaining propulsive movements via the neuromechanical loop to evacuate the colon. In persons with slow-transit constipation (STC), repetitive motor patterns (the term for the human correlate of the CMC) do not increase in frequency after a meal as they do in patients without STC. The reduced or absent postprandial response in persons with STC suggests disrupted extrinsic parasympathetic input to the colon and/or dysfunction in the ENS. When extrinsic nerves are removed by isolating the colon, CMCs occur less frequently in persons with STC than in persons without STC, suggesting that ENS pathophysiology may contribute to motor dysfunction associated with STC. The functional role of the MC is not fully understood, and the specific mechanisms contributing to STC remain unclear. Identifying methods to evoke MCs electrically will provide insight into the mechanisms of the MC and may lead to novel nerve stimulation strategies to induce more efficient colonic motility in patients with chronic constipation.

Stimulating the ENS can directly modulate colonic motility and is an attractive alternative to colectomy for treating chronic constipation. In animal models, diverse stimulation modalities increase motor activity in the colon, including electrically stimulating parasympathetic nerves, electrically stimulating the colon nonspecifically, and optogenetically stimulating specific neurons of the ENS. In patients with chronic constipation, colonic electrical stimulation and sacral nerve stimulation can increase colonic motor patterns. However, the timing parameters to evoke propulsive motor patterns have not been systematically explored and parameter selection relies on empirical testing in the clinical setting. Characterizing the timing constraints of evoked MCs, such as the refractory period and the maximum rate of MC entrainment, will inform neural stimulation strategies to evoke MCs more efficiently and more effectively.

### SUMMARY

Embodiments of the present disclosure include a method of treating gastrointestinal dysmotility in a subject. In accordance with these embodiments, the method includes applying at least one temporal pattern of electrical stimulation to a target nerve or a set of target nerves in a subject having at least one symptom of a gastrointestinal hypermotility disorder and/or a hypomotility disorder. In some embodiments, application of the at least one temporal pattern of electrical stimulation prior to a refractory period suppresses contractions and motility, thereby treating the hypermotility disorder. In some embodiments, application of the at least one temporal pattern of electrical stimulation after a refractory period stimulates contractions and motility, thereby treating the hypomotility disorder.

In some embodiments, the method further comprises selecting the at least one temporal pattern of electrical stimulation based on the subject having one or more symptoms of gastrointestinal hypermotility and/or hypomotility.

In some embodiments, the at least one temporal pattern of electrical stimulation applied prior to the refractory period comprises a continuous pattern of electrical stimulation.

In some embodiments, the at least one temporal pattern of electrical stimulation applied prior to the refractory period comprises a burst pattern of electrical stimulation having an interburst interval less than or equal to the refractory period.

In some embodiments, the at least one temporal pattern of electrical stimulation applied after the refractory period comprises a burst pattern of electrical stimulation.

In some embodiments, the refractory period is determined based on the time between spontaneous gastrointestinal contractions.

In some embodiments, the target nerve or set of target nerves comprise an extrinsic nerve or set of extrinsic nerves, or intrinsic (enteric) nerves. In some embodiments, the extrinsic nerve or set of extrinsic nerves comprise vagal afferent or vagal efferent nerves, splanchnic nerves, pelvic nerves, rectal nerves, lumbar colonic nerves, hypogastric verves, and/or sacral nerves. In some embodiments, the intrinsic nerves comprise nerves that lie within the wall of the gastrointestinal tract. In some embodiments, the extrinsic nerve or set of extrinsic nerves, or the intrinsic (enteric) nerves innervate the gastrointestinal tract.

In some embodiments, the refractory period is determined based on the time between contractions evoked by applied electrical stimulation of extrinsic nerves or intrinsic nerves. In some embodiments, the subject is a human and the refractory period ranges from about 10 seconds to about 60 seconds.

In some embodiments, the continuous pattern of electrical stimulation comprises pulses delivered at a constant frequency for a pre-determined length of time. In some embodiments, the frequency is from about 1 Hz to about 50 Hz. In some embodiments, the pre-determined length of time is from about 1 second to about 60 seconds.

In some embodiments, the burst pattern of electrical stimulation comprises an interburst interval that is greater than the refractory period. In some embodiments, the burst pattern of electrical stimulation comprises bi-phasic pulses. In some embodiments, each phase of the pulses within the burst pattern of electrical stimulation is from about 50 us to about 1000 μs. In some embodiments, the burst pattern of electrical stimulation comprises about 50 to about 150 pulses per burst. In some embodiments, the burst pattern of electrical stimulation comprises an intraburst pulse repetition frequency from about 1 Hz to about 50 Hz. In some embodiments, the burst pattern of electrical stimulation comprises a burst duration from about 1 second to about 60 seconds.

In some embodiments, the subject is a human.

In some embodiments, the at least one symptom of gastrointestinal hypermotility comprises early satiety, nausea, vomiting, bloating, diarrhea, constipation and/or involuntary weight loss.

In some embodiments, the at least one symptom of gastrointestinal hypomotility comprises nausea, vomiting, abdominal pain, abdominal swelling (distention) and/or constipation.

Embodiments of the present disclosure also include a method of treating gastrointestinal hypermotility. In accordance with these embodiments, the method includes applying a continuous pattern of electrical stimulation to a target nerve or set of target nerves in a subject having at least one symptom of an intestinal hypermotility disorder. In some embodiments, the continuous pattern of electrical stimulation is applied prior to a refractory period, thereby suppressing contractions and motility.

Embodiments of the present disclosure also include a method of treating gastrointestinal hypermotility. In accordance with these embodiments, method includes applying a burst pattern of electrical stimulation to a target nerve or set of target nerves in a subject having at least one symptom of an intestinal hypermotility disorder. In some embodiments, the burst pattern of electrical stimulation is applied prior to a refractory period and comprises an interburst interval less than or equal to the refractory period, thereby suppressing contractions and motility.

Embodiments of the present disclosure also include a method of treating gastrointestinal hypomotility. In accordance with these embodiments, the method includes applying a burst pattern of electrical stimulation to a target nerve or set of target nerves in a subject having at least one symptom of a gastrointestinal hypomotility disorder. In some embodiments, the burst pattern of electrical stimulation is applied after a refractory period, thereby stimulating contractions and motility.

Embodiments of the present disclosure also include a method of treating gastrointestinal dysmotility in a subject. In accordance with these embodiments, the method includes programming a pulse generator to output at least one temporal pattern of electrical stimulation to a target nerve or set of target nerves in a subject having at least one symptom of a gastrointestinal hypermotility disorder and/or a hypomotility disorder, and delivering the at least one temporal pattern of electrical stimulation to the subject prior to a refractory period to suppress contractions and motility, thereby treating the hypermotility disorder, and/or delivering the at least one temporal pattern of electrical stimulation to the subject after a refractory period to stimulate contractions and motility, thereby treating the hypomotility disorder.

In some embodiments, the at least one temporal pattern of electrical stimulation applied prior to the refractory period comprises a continuous pattern of electrical stimulation.

In some embodiments, the at least one temporal pattern of electrical stimulation applied prior to the refractory period comprises a burst pattern of electrical stimulation having an interburst interval less than or equal to the refractory period.

In some embodiments, the at least one temporal pattern of electrical stimulation applied after the refractory period comprises a burst pattern of electrical stimulation.

In some embodiments, the at least one temporal pattern of electrical stimulation is delivered to a single subject at one or more time points.

### BRIEF DESCRIPTION OF THE DRAWINGS

FIGS. 1A-1D: Maintained physiological distension drives spontaneous cyclic MCs. (A) Schematic of the isolated colon configuration with distension by an intraluminal rod. (B) Representative recording of spontaneous cyclic MCs (Ba & Bd) and a single MC (Bc & Bd) in AC-coupled (red) and DC-coupled (black) traces with slow wave, pre-complex hyperpolarization, and subthreshold EJPs (a-d). (C) Spontaneous cyclic MCs are abolished by the administration of hexamethonium (300 μM) to the Krebs solution (arrow) (a-b). (D) Subthreshold EJPs are absent after 3 μM atropine is administered to a perfused Krebs solution (arrow) (a-d).

FIGS. 2A-2C: Electrical stimulation evokes premature MCs. (A) Representative recording of evoked MCs (Aa & Ab) and a single evoked MC inset (Ac & Ad) in AC-coupled (red) and DC-coupled (black) traces. (B) The interval preceding spontaneous (x) and evoked (o) MCs from 7 isolated colons. (C) The average interval for each preparation from spontaneous (x) and evoked (o) MCs. The difference in average interval between spontaneous and evoked MCs is statistically significant by paired t-test (p=0.0002, n=7).

FIGS. 3A-3E: Refractory period of the evoked MC. Representative recordings of evoked MCs after spontaneous (A) and after evoked (B) MCs. In red and black are AC-coupled and DC-coupled traces, respectively. Stimulus trains are delivered by a closed-loop controller indicated by arrows. Black fill and white fill arrows indicate stimulation trains that did or did not evoke complexes, respectively. Detection of the beginning and end of complexes are indicated by blue lines. (C) The difference in refractory period at stimulation threshold after spontaneous (x) versus evoked (o) MCs is not statistically significant by paired t-test (p=n=6). (D) The refractory period after spontaneous MCs as a function of approximate stimulation amplitude. The difference in refractory period after spontaneous MCs evoked at threshold versus suprathreshold is statistically significant by paired t-test (p=0.0042, n=7). (E) The refractory period as a function of stimulation amplitude normalized to stimulation threshold (T) after spontaneous (x) or evoked (o) MCs. Outliers (red) at threshold or 1.4× threshold determined by Huber M-Estimation did not contribute to the fitted single-phase exponential decay (blue, R²=0.71). Fitted equation: ŷ=(a−c)·exp(−b·x)+c where ŷ is the estimated refractory period and x is the amplitude. Estimates and 95% CI are: a, 48000 (−42000, 51000); b, 9.0 (−0.8, 18.9); and c, 4.3 (3.0, 5.6).

FIGS. 4A-4D: Closed-loop stimulation repeatedly evokes MCs. (A) Representative recordings of repeatedly evoked MCs with (Aa, red) AC-coupled and (Ab, black) DC-coupled traces. Stimulus trains are delivered by a closed-loop controller indicated by arrows. Black fill and white fill arrows indicate stimulation trains that did or did not evoke complexes, respectively. Detection of the beginning and end of complexes are indicated by blue lines. (B) The number of consecutively evoked CMCs before failing to evoke a CMC as a function of the delay between stimulus onset and the end of the preceding complex normalized the approximation of refractory period (R) in each preparation. Preparations that met exclusions criteria (n=1) are shown in red dashed lines. The number of evoked MCs is significantly greater at a high delay period determined by a paired t-test (p=0.0016, n=7). (C) The duration of entrainment as a function of the delay normalized the approximation of refractory period (R) in each preparation. Preparations that met exclusions criteria (n=1) are shown in red dashed lines. The duration of capture is significantly greater at a high delay period determined by a paired t-test (p=0.0043, n=7). (D) The probability of successfully evoking an MC during repeated closed-loop stimulation split categorically as low (black) and high (red) delay (n=7).

FIGS. 5A-5C: Fluid distension evokes propagating contractions and MCs. (A) Schematic of the isolated colon configuration and dynamic fluid distension. (B) Representative spatiotemporal map of the relative diameter of the colon and overlaid AC-coupled recordings with a propagating contraction and MC evoked by fluid distension. (C) Fluid distension does not evoke a contraction or MC in hexamethonium (300 μM).

FIGS. 6A-6C: Electrical stimulation temporarily suppresses contraction propagation. Representative spatiotemporal diameter-maps of propagating contractions and MCs evoked by fluid distension are temporarily paused by electrical stimulation in the (A) proximal, (B) middle, and (C) distal colon.

FIGS. 7A-7B: (A) Electrical stimulation delivered for 10 s arrested propagation for 10 s in representative spatiotemporal diameter-map. (B) Representative spatiotemporal diameter-map of propagating contractions and MCs evoked by fluid distension are not temporarily paused by electrical stimulation if stimulation is delivered too early.

FIGS. 8A-8F: Propagation velocity is increased after electrical stimulation. Contraction propagation paths from (A) unstimulated and (B) proximal, (C) middle, (D) distal stimulated colons. (E) Representative path (black) and approximate actual (red) and apparent (blue) contraction velocity. (F) Apparent (x) and actual (o) velocity under different stimulation conditions. The ratio of apparent-to-actual velocity during proximal, middle, and distal stimulation is significantly different from the ratio of apparent-to-actual velocity during sham stimulation by one-way ANOVA and Dunnett's comparison with control with subject included as a random effect (n>9). ANOVA F-statistic=12.92 and p=0.00002, and Dunnett's comparison adjusted p-values between proximal, middle, distal and control (sham) are 0.00017, and 0.00021, respectively.

### DETAILED DESCRIPTION

Functional gastrointestinal and motility disorders (FGIMD) are the most common gastrointestinal (GI) disorders in the general population and impact about 1 in 5 persons in the U.S. FGIMD is a group of disorders classified by GI symptoms, including irritable bowel syndrome, fecal incontinence, constipation, and others Patients with FGIMD account for about 40% of the GI problems seen by doctors and therapists. Despite the prevalence and severity of FGIMD, pharmaceutical interventions are largely unsuccessful. Traditional pharmaceuticals, such as opioids, calcium-channel blockers and antimuscarinics, impede gut motility. Electrical nerve stimulation is an alternative treatment.

Peripheral nerve stimulation can relieve gastroparesis, fecal incontinence, and inflammatory bowel disease. Sacral nerve stimulation (SNS) is a particularly promising treatment for lower GI motility disorders because the sacral nerves directly innervate the ileum, colon, and rectum, thus reducing the risk of off-target and side effects. SNS has already been widely used to treat fecal incontinence with mixed results. However, the efficacy of SNS to relieve constipation is limited. The mechanisms of SNS are unknown, and stimulation parameters, or the “therapeutic dose” for SNS are chosen non-systematically. For example, SNS for constipation and fecal incontinence, diseases with opposite motility symptoms, currently employ identical stimulation parameters in hopes of producing opposite effects.

The potential efficacy of nerve stimulation to treat FGIMD is limited by poor understanding of the mechanisms and lack of rationale for the selection of electrical stimulation parameters. As described further herein, embodiments of the present disclosure provide temporal patterns of nerve stimulation to treat FGIMD. These embodiments arose from experimental observations that continuous electrical nerve stimulation resulted in arrest of colonic motility, while burst patterns of electrical stimulation evoked colonic motility. Further, the characteristics of the burst patterns can be selected based upon measurement of the refractory period to evoke colonic motility to ensure that indeed colonic contractions were evoked and that the effects were persistent during continued stimulation.

The results of the present disclosure demonstrate that continuous stimulation (or burst stimulation with a short interburst interval) can be more effective at treating hypermotility disorders by arresting propagation, while burst stimulation with longer interburst intervals, based upon the refractory properties of evoked colonic motor complexes, can be more effective at treating hypo-motility disorders.

One objective of the present disclosure was to quantify the effects of exogenous electrical stimulation on MCs, including both evoking de novo MCs or suppressing MC propagation. In vitro measurements were conducted in the whole mouse colon to characterize the timing constraints of electrically-evoked MCs and identify timing required to suppress propagating MCs with electrical stimulation. Previous work demonstrated that electrical stimulation could evoke MCs prematurely during spontaneous, cyclic MCs; however, in this study, there was no propulsion of content in the lumen, and the study did not provide insight into the refractory properties of the MC cycle or identify methods to induce MCs most effectively. Therefore, as described further herein, the relative and absolute refractory periods of spontaneous and evoked MCs were measured when colonic distension was applied from the lumen, not using isometric force transducers applied to the serosa. It was hypothesized that electrical stimulation applied to specific sites along the colon might disrupt coordination and thus block MC propagation. The results of the present disclosure demonstrated that electrical stimulation delayed, but did not disrupt MCs, once they had been elicited by physiological distension.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

“Entrain” or “entrainment” as used herein refers to a process of altering a subject's biological rhythm to assume a different cycle or frequency. “Entrain” or “entrainment” as used herein also refers to altering a biological rhythm that is symptomatic of disease to match the frequency of applied patterns of electrical stimulation to treat one or more symptoms of the disease.

“Gastrointestinal tract motility” or “gut motility” as used herein refers to the motility and contractions of the digestive system and the transit of the contents within it. Accordingly, when nerves and/or muscles in any portion of the digestive tract do not function normally (e.g., hypermotility or hypomotility), a subject can develop one or more symptoms related to guy dysmotility.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human) In some embodiments, the subject may be a human or a non-human. In one embodiment, the subject is a human. The subject or patient may be undergoing various forms of treatment.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. METHODS OF TREATMENT

Embodiments of the present disclosure provide important new insights as to how to use electrical stimulation to increase gastrointestinal motility and transit in the large intestine of a subject. The major objectives of the present disclosure include the application of electrical nerve stimulation to the ENS during the MC cycle to: (i) quantify the refractory period, (ii) inform and evaluate closed-loop stimulation that allowed for repetitively evoking MCs, and (iii) identify methods to suppress MC propagation. Whilst the interval between MCs and their duration varied between isolated mouse colons, the findings of the present disclosure demonstrate temporal considerations that are important for gastrointestinal electrical stimulation as a technique to increase propulsive contraction and motility in the gastrointestinal tract, which is largely achieved by reducing the interval between MCs while accounting for the refractory period.

Circumferential stretch of the colon stimulates the ENS and has a major influence on determining the physiological rate of MCs. Loss of major parts of the ENS leads to major dysfunction of MC activity. In humans, the rate of MCs fail to increase in a postprandial response, which likely contributes to slowed colonic transit in persons with STC. Thus, treatments are desired to evoke MCs and increase the rate of MCs in persons with STC. Direct electrical stimulation of the colon evokes MCs and was used to pace the colon electrically and increase the rate of MCs in attempt to treat colonic dysmotility. However, the timing constraints that limit the rate of MCs had not been characterized previously.

Results of the present disclosure demonstrate that the responses to electrical nerve stimulation are highly dependent on timing of the stimulus, relative to ongoing activity in the colon. The measurements of the duration of MCs, the interval between MCs, and the ability to evoke MCs prematurely were consistent with previous in vitro results. In addition to evoking MCs with electrical stimulation, the hypothesis that occluding MC propagation by electrically stimulating the colon was also tested. Although, MC propagation was not completely arrested, MC propagation was temporarily halted for the duration of electrical stimulation. The ongoing colonic activity should be taken into account to evoke or suppress effectively colonic motor patterns. Closed-loop stimulation or predictive models would improve real-time treatments for motility disorders in the colon.

Refractory period. As described further herein, the ability to evoke MCs with exogenous stimulation was dependent on the timing of stimulation relative to prior spontaneous or evoked MCs. As stimulation amplitude increased, the MC refractory period decreased to an absolute refractory period. The mechanisms underlying the MC refractory period are unknown. The interval between MCs in the isolated mouse colon decreases significantly in the presence of the nitric oxide synthase (NOS) inhibitor, N-nitro-L-arginine (L-NNA), and the interval increases significantly in the presence of the NOS substrate, L-arginine. Therefore, inhibition by nitric oxide is likely involved in the refractory mechanisms of the MC, but further experimentation is necessary to test this hypothesis. In the absence of pharmacological intervention, the minimum interval between MCs in the isolated mouse colon is ˜30 s, when the colon is distended by multiple fecal pellets. The refractory period of MCs evoked by electrical stimulation is much lower than the physiological interval between MCs, which is influenced by distension and extrinsic nerve input under physiological conditions. However, describing the minimal delay necessary to evoke an MC as a refractory period is not entirely accurate, as the MC is not necessarily a binary event. For example, an action potential in a nerve fiber is a binary event that has a refractory period caused by the inactivation of voltage-gated sodium channels. The MC is not well described as a binary event. In the present disclosure, an MC was treated as an event if it met the criteria defined by an online detection algorithm: frequency content between 1 and 5 Hz above a user-defined threshold and sustained for a 3 s interval. From the perspective of the detection algorithm, a 6 s long MC was equivalent to a 30 s long MC. Thus, an assumption of the present disclosure is that all MCs are identical, and the recording site can be categorized at any given time as “during an ongoing MC” or “during a quiescent period.”

Entrainment. Closed-loop electrical stimulation was employed to entrain cyclic MC events, similar to achieving capture in cardiac pacing. Cardiac pacing intends to reset the rhythm of the heart by electrical stimulation, and cardiac capture is achieved in open or closed-loop systems that confirm the pacing stimulus leads to depolarization of the ventricles. Colonic pacing by direct electrical stimulation has been used experimentally to treat colonic dysmotility. Previous applications of colonic pacing have been open-loop systems with continuous stimulation at a pre-determined frequency. In the present disclosure, temporary colonic entrainment was achieved in a closed-loop system of colonic pacing to evoke and record MCs. Despite the limitations of using an isolated whole mouse colon, the absolute refractory period is a practical minimum interval between attempts to evoke MCs.

The presence of a minimum interval between evoked MCs suggests that bursting stimulation patterns may more efficiently increase colonic motility than continuous stimulation. Continuous stimulation is the predominant pattern of colonic electrical stimulation and sacral nerve stimulation. For example, previous studies treated STC in two patients who had failed to respond to conventional therapies with colonic electrical stimulation using 150 μs pulses and 10 pulses per second applied continuously for 2 min intervals repeated every 20 min Another study treated constipation due to colonic inertia in three out of nine patients using 200 ms pulses applied continuously at a frequency 15% higher than electrical slow wave frequency. The mean duration of repetitive motor patterns in colons isolated from persons without STC is 51.5 s, and the mean duration of repetitive motor patterns recorded in vivo from persons without STC is 10.4 s. However, the physiological duration of repetitive motor patterns has not been used to inform stimulation parameters to treat constipation. It was predicted that effective stimulation parameters will employ bursts of stimulation delivered at an interval equal to the MC duration, as it occurs in vivo, plus twice the refractory period, based on the minimum interval between evoked MCs. In the absence of a quantification of the refractory period in humans, it was assumed that the refractory period of the evoked MC scales between the mouse and the human as the MC duration scales between isolated mouse colon and isolated human colon. The duration of MCs in isolated human colons is about 2.1 times greater than in isolated mouse colons. Therefore, the optimal interval between bursts of electrical stimulation to evoke repetitive motor patterns in patients with STC is 28.3 s.

Interrupting propagating MCs. In addition to evoking MCs, electrical stimulation could temporarily arrest MC propagation. After stimulation ceased, propagation resumed, and the velocity of propagation increased. The increase in velocity following cessation of electrical stimulation decreased as the stimulation site move from proximal, to middle, to distal colon. MC propagation velocity was thus slowed more by temporarily suppressing propagation in the distal colon at the location of greatest velocity than it was by suppressing propagation at the location of least velocity. While this may be caused by physiological differences along the colon, it is likely that the differences are an artifact of the preparation because MCs evoked by fluid distension typically accelerate along the isolated colon. The increase in velocity was observed after the electrical stimulation was delivered, which was initiated when the propagating MC arrives at the stimulation site. As the stimulation site was moved aborally along the colon, there was less remaining distance for the MC to propagate. Further, the propagating MC increased in velocity as it traveled aborally, and it was moving fastest in the distal colon. In other words, the decrease in actual velocity following electrical stimulation in the distal colon compared to the proximal colon could be an effect of physiology, mechanical properties, or a combination thereof.

Suppressing MC propagation was sensitive to the timing of electrical stimulation. Stimulation must be delivered just as the contraction wavefront was about to reach the stimulation site, otherwise the contraction will continue past the stimulation site unimpeded, and this observation illustrates the challenge of reliably suppressing MCs in vivo. Temporarily halting MCs may provide future insights into the processes that support MC propagation.

One goal of the present disclosure included quantifying the effects of the timing of electrical stimulation on modulation of MCs, including both entraining MCs or temporarily suppressing MC propagation. The relative and absolute refractory period of the MC was measured in the isolated whole mouse colon and used the refractory period to design a closed-loop stimulation paradigm to evoke MCs at a maximal rate. Colonic entrainment began to fail after several minutes and increasing the delay between stimulation and the preceding MC nearly doubled the duration of successful entrainment. Electrical stimulation could temporarily halt MC propagation and propagation velocity subsequently increased after cessation of stimulation. These provide design criteria for electrical stimulation parameters (e.g., delivering bursts of electrical stimulation at an interval of 28.3 s to entrain repetitive motor patterns efficiently in patients with constipation). These neuromodulation design strategies may more efficiently and effectively evoke MCs in treating STC to treat colonic motility disorders.

In accordance with the above, embodiments of the present disclosure include methods of treating gastrointestinal dysmotility in a subject. In some embodiments, the method includes applying at least one temporal pattern of electrical stimulation to a target nerve or a set of target nerves in a subject having at least one symptom of a gastrointestinal hypermotility disorder and/or a hypomotility disorder. In some embodiments, application of the temporal pattern of electrical stimulation prior to a refractory period suppresses contractions and motility, which results in the treatment and/or prevention of a hypermotility disorder. In other embodiments, application of the at least one temporal pattern of electrical stimulation after a refractory period stimulates contractions and motility, which results in the treatment and/or preventions of a hypomotility disorder. The aforementioned methods of treating and/or preventing a hypermotility disorder and/or a hypomotility disorder can include administering the treatment separately to different individuals who suffer from a hypermotility disorder or a hypomotility disorder. In other embodiments, methods of treating and/or preventing a hypermotility disorder and/or a hypomotility disorder can include administering the treatment to a single individual suffering from symptoms of both a hypermotility disorder and a hypomotility disorder at different points in time (e.g., applying electrical stimulation from a single implantable medical device at different times).

In some embodiments, the method includes selecting at least one temporal pattern of electrical stimulation to be administered, based on whether a subject has one or more symptoms of gastrointestinal hypermotility and/or hypomotility. If the subject has been diagnosed with, or is suffering from, a hypermotility disorder or condition, then the temporal pattern of electrical stimulation that is applied prior to the refractory period is a continuous pattern of electrical stimulation, or a burst pattern of electrical stimulation with an interburst interval less than or equal to the refractory period. As described further herein, this method results in the suppression of contractions and motility in the subject's gastrointestinal tract. Alternatively, if the subject has been diagnosed with, or is suffering from, a hypomotility disorder or condition, then the temporal pattern of electrical stimulation that is applied after the refractory period is a burst pattern of electrical stimulation with an interburst interval greater than the refractory period. As described further herein, this method results in the stimulation of contractions and motility in the subject's gastrointestinal tract.

In some embodiments, the target nerve or set of target nerves includes an extrinsic nerve or set of extrinsic nerves. In some embodiments, the target nerve or set of target nerves includes an intrinsic (enteric) nerve or set of intrinsic (enteric) nerves. In some embodiments, the extrinsic nerve or set of extrinsic nerves comprise vagal afferent or vagal efferent nerves, splanchnic nerves, pelvic nerves, rectal nerves, lumbar colonic nerves, hypogastric verves, and/or sacral nerves. In other embodiments, the intrinsic nerves comprise nerves that lie within the wall of the gastrointestinal tract. In some embodiments, the extrinsic nerve or set of extrinsic nerves, or the intrinsic (enteric) nerves innervate the gastrointestinal tract.

The ability to evoke myoelectric complexes (MCs) with exogenous stimulation is dependent on the timing of stimulation relative to prior spontaneous or evoked MCs; this is generally referred to as the refractory period. In some embodiments, the refractory period is determined based on the time between spontaneous gastrointestinal contractions. In some embodiments, the refractory period is determined based on the time between contractions evoked by applied electrical stimulation of extrinsic nerves or intrinsic nerves in a subject. In some embodiments, the subject is a human.

In some embodiments, the subject is a human and the refractory period ranges from about 10 seconds to about 60 seconds. In some embodiments, the subject is a human and the refractory period ranges from about 10 seconds to about 55 seconds. In some embodiments, the subject is a human and the refractory period ranges from about 10 seconds to about 50 seconds. In some embodiments, the subject is a human and the refractory period ranges from about 10 seconds to about 45 seconds. In some embodiments, the subject is a human and the refractory period ranges from about 10 seconds to about 40 seconds. In some embodiments, the subject is a human and the refractory period ranges from about 10 seconds to about 35 seconds. In some embodiments, the subject is a human and the refractory period ranges from about 10 seconds to about 30 seconds. In some embodiments, the subject is a human and the refractory period ranges from about 10 seconds to about 25 seconds. In some embodiments, the subject is a human and the refractory period ranges from about 10 seconds to about 20 seconds. In some embodiments, the subject is a human and the refractory period ranges from about 20 seconds to about 60 seconds. In some embodiments, the subject is a human and the refractory period ranges from about 25 seconds to about 60 seconds. In some embodiments, the subject is a human and the refractory period ranges from about 30 seconds to about 60 seconds. In some embodiments, the subject is a human and the refractory period ranges from about 35 seconds to about 60 seconds. In some embodiments, the subject is a human and the refractory period ranges from about 40 seconds to about 60 seconds. In some embodiments, the subject is a human and the refractory period ranges from about 45 seconds to about 60 seconds. In some embodiments, the subject is a human and the refractory period ranges from about 50 seconds to about 60 seconds. In some embodiments, the subject is a human and the refractory period ranges from about 20 seconds to about 50 seconds. In some embodiments, the subject is a human and the refractory period ranges from about 25 seconds to about 45 seconds. In some embodiments, the subject is a human and the refractory period ranges from about 30 seconds to about 40 seconds. In some embodiments, the subject is a human and the refractory period ranges from about 15 seconds to about 55 seconds. In some embodiments, the subject is a human and the refractory period ranges from about 25 seconds to about 50 seconds.

In some embodiments, the temporal pattern of electrical stimulation comprises a continuous pattern of electrical stimulation applied prior to a refractory period, or comprises a burst pattern of electrical stimulation having an interburst interval that is less than or equal to the refractory period (e.g., to treat a gastrointestinal hypermotility disorder). In other embodiments, the temporal pattern of electrical stimulation comprises a burst pattern of electrical stimulation with an interburst interval that is greater than the refractory period (e.g., to treat a gastrointestinal hypomotility disorder).

In some embodiments, the continuous pattern of electrical stimulation or the burst pattern of electrical stimulation having an interburst interval that is less than or equal to the refractory period that is applied to a subject to treat a hypermotility disorder or symptom of a hypermotility disorder is comprised of pulses delivered at a constant frequency for a pre-determined length of time. In some embodiments, the frequency is from about 1 Hz to about Hz. In some embodiments, the frequency is from about 1 Hz to about 45 Hz. In some embodiments, the frequency is from about 1 Hz to about 40 Hz. In some embodiments, the frequency is from about 1 Hz to about 35 Hz. In some embodiments, the frequency is from about 1 Hz to about 30 Hz. In some embodiments, the frequency is from about 1 Hz to about Hz. In some embodiments, the frequency is from about 1 Hz to about 20 Hz. In some embodiments, the frequency is from about 1 Hz to about 15 Hz. In some embodiments, the frequency is from about 1 Hz to about 10 Hz. In some embodiments, the frequency is from about 10 Hz to about 50 Hz. In some embodiments, the frequency is from about 15 Hz to about Hz. In some embodiments, the frequency is from about 20 Hz to about 50 Hz. In some embodiments, the frequency is from about 25 Hz to about 50 Hz. In some embodiments, the frequency is from about 30 Hz to about 50 Hz. In some embodiments, the frequency is from about 35 Hz to about 50 Hz. In some embodiments, the frequency is from about 40 Hz to about Hz. In some embodiments, the frequency is from about 10 Hz to about 40 Hz. In some embodiments, the frequency is from about 20 Hz to about 30 Hz.

In some embodiments, the pre-determined length of time during which the continuous pattern of electrical stimulation is applied is from about 1 second to about 60 seconds. In some embodiments, the pre-determined length of time is from about 1 second to about 55 seconds. In some embodiments, the pre-determined length of time is from about 1 second to about 50 seconds. In some embodiments, the pre-determined length of time is from about 1 second to about 45 seconds. In some embodiments, the pre-determined length of time is from about 1 second to about 40 seconds. In some embodiments, the pre-determined length of time is from about 1 second to about 35 seconds. In some embodiments, the pre-determined length of time is from about 1 second to about 30 seconds. In some embodiments, the pre-determined length of time is from about 1 second to about 25 seconds. In some embodiments, the pre-determined length of time is from about 1 second to about 20 seconds. In some embodiments, the pre-determined length of time is from about 1 second to about 15 seconds. In some embodiments, the pre-determined length of time is from about 1 second to about 10 seconds. In some embodiments, the pre-determined length of time is from about 10 seconds to about 60 seconds. In some embodiments, the pre-determined length of time is from about 15 seconds to about 60 seconds. In some embodiments, the pre-determined length of time is from about 20 seconds to about 60 seconds. In some embodiments, the pre-determined length of time is from about 25 seconds to about 60 seconds. In some embodiments, the pre-determined length of time is from about 30 seconds to about 60 seconds. In some embodiments, the pre-determined length of time is from about 35 seconds to about 60 seconds. In some embodiments, the pre-determined length of time is from about 40 seconds to about 60 seconds. In some embodiments, the pre-determined length of time is from about 45 seconds to about 60 seconds. In some embodiments, the pre-determined length of time is from about 50 seconds to about 60 seconds. In some embodiments, the pre-determined length of time is from about 20 seconds to about 50 seconds. In some embodiments, the pre-determined length of time is from about 30 seconds to about 40 seconds.

In some embodiments, the temporal pattern of electrical stimulation comprises a burst pattern of electrical stimulation. In some embodiments, the burst pattern of electrical stimulation comprises bi-phasic pulses. In some embodiments, each phase of the pulses within the burst pattern of electrical stimulation is from about 50 us to about 1000 μs. In some embodiments, each phase of the pulses within the burst pattern of electrical stimulation is from about 100 us to about 1000 μs. In some embodiments, each phase of the pulses within the burst pattern of electrical stimulation is from about 200 us to about 1000 μs. In some embodiments, each phase of the pulses within the burst pattern of electrical stimulation is from about 300 us to about 1000 μs. In some embodiments, each phase of the pulses within the burst pattern of electrical stimulation is from about 400 us to about 1000 μs. In some embodiments, each phase of the pulses within the burst pattern of electrical stimulation is from about 500 us to about 1000 μs. In some embodiments, each phase of the pulses within the burst pattern of electrical stimulation is from about 600 us to about 1000 μs. In some embodiments, each phase of the pulses within the burst pattern of electrical stimulation is from about 700 us to about 1000 μs. In some embodiments, each phase of the pulses within the burst pattern of electrical stimulation is from about 800 us to about 1000 μs. In some embodiments, each phase of the pulses within the burst pattern of electrical stimulation is from about 900 us to about 1000 μs. In some embodiments, each phase of the pulses within the burst pattern of electrical stimulation is from about 50 us to about 900 μs. In some embodiments, each phase of the pulses within the burst pattern of electrical stimulation is from about 50 us to about 800 μs. In some embodiments, each phase of the pulses within the burst pattern of electrical stimulation is from about 50 us to about 700 μs. In some embodiments, each phase of the pulses within the burst pattern of electrical stimulation is from about 50 μs to about 600 μs. In some embodiments, each phase of the pulses within the burst pattern of electrical stimulation is from about 50 μs to about 500 μs. In some embodiments, each phase of the pulses within the burst pattern of electrical stimulation is from about 50 μs to about 400 μs. In some embodiments, each phase of the pulses within the burst pattern of electrical stimulation is from about 50 μs to about 300 μs. In some embodiments, each phase of the pulses within the burst pattern of electrical stimulation is from about 50 μs to about 200 μs. In some embodiments, each phase of the pulses within the burst pattern of electrical stimulation is from about 50 μs to about 100 μs. In some embodiments, each phase of the pulses within the burst pattern of electrical stimulation is from about 100 μs to about 900 μs. In some embodiments, each phase of the pulses within the burst pattern of electrical stimulation is from about 200 μs to about 800 μs. In some embodiments, each phase of the pulses within the burst pattern of electrical stimulation is from about 400 μs to about 600 μs. In some embodiments, each phase of the pulses within the burst pattern of electrical stimulation is from about 400 μs to about 800 μs. In some embodiments, each phase of the pulses within the burst pattern of electrical stimulation is from about 500 μs to about 1000 μs.

In some embodiments, the burst pattern of electrical stimulation comprises about 50 to about 150 pulses per burst. In some embodiments, the burst pattern of electrical stimulation comprises about 60 to about 150 pulses per burst. In some embodiments, the burst pattern of electrical stimulation comprises about 70 to about 150 pulses per burst. In some embodiments, the burst pattern of electrical stimulation comprises about 80 to about 150 pulses per burst. In some embodiments, the burst pattern of electrical stimulation comprises about 90 to about 150 pulses per burst. In some embodiments, the burst pattern of electrical stimulation comprises about 100 to about 150 pulses per burst. In some embodiments, the burst pattern of electrical stimulation comprises about 110 to about 150 pulses per burst. In some embodiments, the burst pattern of electrical stimulation comprises about 120 to about 150 pulses per burst. In some embodiments, the burst pattern of electrical stimulation comprises about 130 to about 150 pulses per burst. In some embodiments, the burst pattern of electrical stimulation comprises about 140 to about 150 pulses per burst. In some embodiments, the burst pattern of electrical stimulation comprises about 50 to about 140 pulses per burst. In some embodiments, the burst pattern of electrical stimulation comprises about 50 to about 130 pulses per burst. In some embodiments, the burst pattern of electrical stimulation comprises about 50 to about 120 pulses per burst. In some embodiments, the burst pattern of electrical stimulation comprises about 50 to about 110 pulses per burst. In some embodiments, the burst pattern of electrical stimulation comprises about 50 to about 100 pulses per burst. In some embodiments, the burst pattern of electrical stimulation comprises about 50 to about 90 pulses per burst. In some embodiments, the burst pattern of electrical stimulation comprises about 50 to about 80 pulses per burst. In some embodiments, the burst pattern of electrical stimulation comprises about 50 to about 70 pulses per burst. In some embodiments, the burst pattern of electrical stimulation comprises about 50 to about 60 pulses per burst. In some embodiments, the burst pattern of electrical stimulation comprises about 75 to about 125 pulses per burst. In some embodiments, the burst pattern of electrical stimulation comprises about 125 to about 150 pulses per burst. In some embodiments, the burst pattern of electrical stimulation comprises about 100 to about 125 pulses per burst.

In some embodiments, the burst pattern of electrical stimulation comprises an intraburst pulse repetition frequency from about 1 Hz to about 50 Hz. In some embodiments, the burst pattern of electrical stimulation comprises an intraburst pulse repetition frequency from about 5 Hz to about 50 Hz. In some embodiments, the burst pattern of electrical stimulation comprises an intraburst pulse repetition frequency from about 10 Hz to about 50 Hz. In some embodiments, the burst pattern of electrical stimulation comprises an intraburst pulse repetition frequency from about 15 Hz to about 50 Hz. In some embodiments, the burst pattern of electrical stimulation comprises an intraburst pulse repetition frequency from about 20 Hz to about 50 Hz. In some embodiments, the burst pattern of electrical stimulation comprises an intraburst pulse repetition frequency from about 25 Hz to about 50 Hz. In some embodiments, the burst pattern of electrical stimulation comprises an intraburst pulse repetition frequency from about 30 Hz to about 50 Hz. In some embodiments, the burst pattern of electrical stimulation comprises an intraburst pulse repetition frequency from about 35 Hz to about 50 Hz. In some embodiments, the burst pattern of electrical stimulation comprises an intraburst pulse repetition frequency from about 40 Hz to about 50 Hz. In some embodiments, the burst pattern of electrical stimulation comprises an intraburst pulse repetition frequency from about 45 Hz to about 50 Hz. In some embodiments, the burst pattern of electrical stimulation comprises an intraburst pulse repetition frequency from about 1 Hz to about 45 Hz. In some embodiments, the burst pattern of electrical stimulation comprises an intraburst pulse repetition frequency from about 1 Hz to about 40 Hz. In some embodiments, the burst pattern of electrical stimulation comprises an intraburst pulse repetition frequency from about 1 Hz to about 35 Hz. In some embodiments, the burst pattern of electrical stimulation comprises an intraburst pulse repetition frequency from about 1 Hz to about 30 Hz. In some embodiments, the burst pattern of electrical stimulation comprises an intraburst pulse repetition frequency from about 1 Hz to about 25 Hz. In some embodiments, the burst pattern of electrical stimulation comprises an intraburst pulse repetition frequency from about 1 Hz to about 20 Hz. In some embodiments, the burst pattern of electrical stimulation comprises an intraburst pulse repetition frequency from about 1 Hz to about 15 Hz. In some embodiments, the burst pattern of electrical stimulation comprises an intraburst pulse repetition frequency from about 1 Hz to about 10 Hz. In some embodiments, the burst pattern of electrical stimulation comprises an intraburst pulse repetition frequency from about 1 Hz to about 5 Hz. In some embodiments, the burst pattern of electrical stimulation comprises an intraburst pulse repetition frequency from about 20 Hz to about 40 Hz. In some embodiments, the burst pattern of electrical stimulation comprises an intraburst pulse repetition frequency from about 10 Hz to about 30 Hz. In some embodiments, the burst pattern of electrical stimulation comprises an intraburst pulse repetition frequency from about 15 Hz to about 45 Hz.

In some embodiments, the burst pattern of electrical stimulation comprises a burst duration from about 1 second to about 60 seconds. In some embodiments, the burst pattern of electrical stimulation comprises a burst duration from about 5 second to about 60 seconds. In some embodiments, the burst pattern of electrical stimulation comprises a burst duration from about 10 second to about 60 seconds. In some embodiments, the burst pattern of electrical stimulation comprises a burst duration from about 15 second to about 60 seconds. In some embodiments, the burst pattern of electrical stimulation comprises a burst duration from about 20 second to about 60 seconds. In some embodiments, the burst pattern of electrical stimulation comprises a burst duration from about 25 second to about 60 seconds. In some embodiments, the burst pattern of electrical stimulation comprises a burst duration from about 30 second to about 60 seconds. In some embodiments, the burst pattern of electrical stimulation comprises a burst duration from about 35 second to about 60 seconds. In some embodiments, the burst pattern of electrical stimulation comprises a burst duration from about 40 second to about 60 seconds. In some embodiments, the burst pattern of electrical stimulation comprises a burst duration from about 45 second to about 60 seconds. In some embodiments, the burst pattern of electrical stimulation comprises a burst duration from about 50 second to about 60 seconds. In some embodiments, the burst pattern of electrical stimulation comprises a burst duration from about 55 second to about 60 seconds. In some embodiments, the burst pattern of electrical stimulation comprises a burst duration from about 1 second to about 55 seconds. In some embodiments, the burst pattern of electrical stimulation comprises a burst duration from about 1 second to about 50 seconds. In some embodiments, the burst pattern of electrical stimulation comprises a burst duration from about 1 second to about 45 seconds. In some embodiments, the burst pattern of electrical stimulation comprises a burst duration from about 1 second to about 40 seconds. In some embodiments, the burst pattern of electrical stimulation comprises a burst duration from about 1 second to about 35 seconds. In some embodiments, the burst pattern of electrical stimulation comprises a burst duration from about 1 second to about 30 seconds. In some embodiments, the burst pattern of electrical stimulation comprises a burst duration from about 1 second to about 25 seconds. In some embodiments, the burst pattern of electrical stimulation comprises a burst duration from about 1 second to about 20 seconds. In some embodiments, the burst pattern of electrical stimulation comprises a burst duration from about 1 second to about 15 seconds. In some embodiments, the burst pattern of electrical stimulation comprises a burst duration from about 1 second to about 10 seconds. In some embodiments, the burst pattern of electrical stimulation comprises a burst duration from about 1 second to about 5 seconds. In some embodiments, the burst pattern of electrical stimulation comprises a burst duration from about 10 second to about 50 seconds. In some embodiments, the burst pattern of electrical stimulation comprises a burst duration from about 10 second to about 40 seconds. In some embodiments, the burst pattern of electrical stimulation comprises a burst duration from about 20 second to about 40 seconds. In some embodiments, the burst pattern of electrical stimulation comprises a burst duration from about 30 second to about 50 seconds.

In accordance with these embodiments, the present disclosure includes methods of treating gastrointestinal dysmotility in a subject. In some embodiments, the subject is a human. In some embodiments, treating gastrointestinal dysmotility includes treating one or more symptoms of gastrointestinal hypermotility, including but not limited to, early satiety, nausea, vomiting, bloating, diarrhea, constipation, involuntary weight loss, and any combination thereof. In some embodiments, treating gastrointestinal dysmotility includes treating one or more symptoms of gastrointestinal hypomotility, including but not limited to, nausea, vomiting, abdominal pain, abdominal swelling (distention), constipation, and any combination thereof.

### 3. NEUROMODULATION SYSTEMS

As described further herein, electrical neuromodulation is an attractive approach for alleviating dysmotility in the gastrointestinal tract, such as, for example, gastric electrical stimulation for the treatment of delayed gastric emptying or sacral nerve stimulation for the treatment of fecal incontinence. However, further advancement in neuromodulation techniques for gastrointestinal dysmotility has been hindered by incomplete understanding of the effects of stimulation parameters and the timing considerations for controlling motility in the gastrointestinal of a subject. For example, altering parameters in sacral nerve stimulation improves outcomes in some patients with bowel dysfunction. Without understanding the limiting factors of evoked colonic activity, attempts to evoke colonic activity more efficiently are not grounded in physiology and are limited to proceed in a trial and error fashion. It was hypothesized that the likelihood of evoking consecutive colonic MCs may be limited by a refractory period, and this should be an important consideration in designing electrical stimulation to increase coordinated motility in the colon. Results of the present disclosure demonstrated that the MC in the isolated mouse colon has a relative and absolute refractory period, suggesting subsequent MCs cannot successfully be evoked less than 4 s after the preceding MC. Further, increasing the delay between evoked MCs increases the duration of successfully evoked MCs. Results of the present disclosure demonstrated that timing considerations of evoked motor patterns impact the efficacy of modulating colonic motility, and bursting patterns of stimulation may be more effective than continuous stimulation. These findings have implications in neuromodulation of viscera function, particularly in cases of colonic dysmotility and sacral nerve stimulation for bowel dysfunction.

In accordance with this, embodiments of the present disclosure include methods of treating gastrointestinal hypermotility and hypomotility conditions in a subject by applying electrical stimulation. In some embodiments, the method includes treating a human subject having at least one symptom of an intestinal hypermotility disorder by applying a continuous pattern of electrical stimulation to a target nerve or set of target nerves. In some embodiments, the continuous pattern of electrical stimulation is applied prior to a refractory period, thereby suppressing contractions and motility. In other embodiments, a burst pattern of electrical stimulation is applied prior to a refractory period and comprises an interburst interval less than or equal to the refractory period, thereby suppressing contractions and motility. In some embodiments, the method includes treating a human subject having at least one symptom of a gastrointestinal hypomotility disorder by applying a burst pattern of electrical stimulation to a target nerve or set of target nerves. In some embodiments, the burst pattern of electrical stimulation is applied after a refractory period, thereby stimulating contractions and motility. In some embodiments, the burst pattern of electrical stimulation comprises an interburst interval that is greater than the refractory period.

Embodiments of the present disclosure also include methods of treating gastrointestinal dysmotility by programming a pulse generator to output at least one temporal pattern of electrical stimulation to a target nerve or set of target nerves in a subject having at least one symptom of a gastrointestinal hypermotility disorder and/or a hypomotility disorder. In some embodiments, the method includes delivering at least one temporal pattern of electrical stimulation to the subject prior to a refractory period to suppress contractions and motility, thereby treating the hypermotility disorder. In some embodiments, the at least one temporal pattern of electrical stimulation applied prior to the refractory period is a continuous pattern of electrical stimulation. In some embodiments, the at least one temporal pattern of electrical stimulation applied prior to the refractory period is a burst pattern of electrical stimulation having an interburst interval that is less than or equal to a refractory period. In other embodiments, the method includes delivering the at least one temporal pattern of electrical stimulation to the subject after a refractory period to stimulate contractions and motility, thereby treating the hypomotility disorder. In some embodiments, the at least one temporal pattern of electrical stimulation applied after the refractory period is a burst pattern of electrical stimulation having an interburst interval that is greater than a refractory period.

The aforementioned methods of treating and/or preventing a hypermotility disorder and/or a hypomotility disorder can include administering the treatment separately to different individuals who suffer from a hypermotility disorder or a hypomotility disorder. In other embodiments, methods of treating and/or preventing a hypermotility disorder and/or a hypomotility disorder can include administering the treatment to a single individual suffering from symptoms of both a hypermotility disorder and a hypomotility disorder at different points in time (e.g., applying electrical stimulation from a single implantable medical device at different times).

In accordance with these embodiments, methods of the present disclosure also include operating an implantable neuromodulation device to treat gastrointestinal dysmotility in a subject. In some embodiments, the methods include configuring a neuromodulation device to apply a temporal pattern of electrical stimulation to a target nerve or set of target nerves to treat one or more symptoms of gastrointestinal dysmotility in the subject. In some embodiments, methods of modulating contractions and motility in the gastrointestinal tract of a subject using the neuromodulation device include treating one or more symptoms of a gastrointestinal and/or motility disorder in the subject. As described further herein, treating gastrointestinal dysmotility using the methods of the present disclosure includes treating one or more symptoms of gastrointestinal hypermotility, including but not limited to, early satiety, nausea, vomiting, bloating, diarrhea, constipation, involuntary weight loss, and any combination thereof. In some embodiments, treating gastrointestinal dysmotility includes treating one or more symptoms of gastrointestinal hypomotility, including but not limited to, nausea, vomiting, abdominal pain, abdominal swelling (distention), constipation, and any combination thereof.

In some embodiments, an implantable neuromodulation device to treat gastrointestinal dysmotility disorder in a subject can include a neuromodulation system comprising one or more implantable electrodes and a signal generator device. In some embodiments, the system further comprises electrical terminals configured for being respectively coupled to a plurality of electrodes implanted within tissue (e.g., gastrointestinal tissue), analog output circuitry configured for delivering therapeutic electrical energy between the plurality of electrical terminals in accordance with a set of modulation parameters that includes a defined current value (e.g., a user-programmed value), and a voltage regulator configured for supplying an adjustable compliance voltage to the analog output circuitry. The neuromodulation device and/or system can further comprises control/processing circuitry configured for performing a compliance voltage calibration process at a compliance voltage adjustment interval by periodically computing an adjusted compliance voltage value as a function of a compliance voltage margin, directing the voltage regulator to adjust the compliance voltage to the adjusted compliance voltage value, and for adjusting at least one of the compliance voltage adjustment interval and the compliance voltage margin during the voltage compliance calibration process. The compliance voltage adjustments may be automatically performed as described above or manually performed in response to user input.

### 4. MATERIALS AND METHODS

Ethical approval. All procedures were approved by the Animal Welfare Committee of Flinders University or the Institutional Animal Care and Use Committee of Duke University. Wild-type C57BL/6 (n=32) mice of either sex between 6 and 10 weeks of age and between 17 and 29 grams were housed in same-sex cages with four to five mice per cage. Mice were given free access to food (5053 PicoLab, Lab Diet, St. Louis, MO, USA or Mouse Breeder's Diet, Gordon's Specialty Stock Feeds, Yanderra, N. S. W., Australia) and water and maintained on a semi-diurnal lighting cycle. All mice were euthanized by cervical dislocation and decapitation under isoflurane anesthesia in accordance with ethics approvals.

The whole colon was dissected from each mouse and kept at 36° C. in Krebs solution bubbled with 5% CO₂/95% O₂. The Krebs solution contained (mM): 118 NaCl, 4.7 KCl, 1.0 NaH₂PO₄, 25 NaHCO₃, 1.2 MgCl₂, 11 d-glucose, and 2.5 CaCl₂) and was prepared fresh daily. The whole colon was preserved to maintain the integrity of intrinsic circuitry, whilst the extrinsic nerves were dissected away. The content of the colon was allowed to empty, assisted by gently flushing with warm Krebs solution. Myoelectric activity was recorded in the isolated mouse colon under two experimental configurations: maintained physiological distension or intraluminal Krebs perfusion.

Refractory period and entrainment experimental design. The refractory period was first measured, and subsequently the properties of MC entrainment, in the same isolated mouse colons with maintained physiological distension. In each preparation, spontaneous cyclic MCs were recorded prior to conducting any interventions. The order of measurements was not randomized because the refractory period informed the settings used to entrain MCs. The investigator was not blinded to stimulus amplitude or delay, and the same investigator performed data analyses. Five parameters were measured: the refractory period with stimulation amplitude equal to the threshold to evoke an MC (i) after a spontaneous MC and (ii) after an evoked MC, (iii) the refractory period after a spontaneous MC with stimulation amplitude equal to approximately 140% of threshold, and the duration of MC entrainment with delay approximately equal to (iv) the refractory period and (v) twice the refractory period. Preparations in which electrical stimulation delivered at threshold and 30 s after the end of the preceding MC did not evoke an MC were excluded (n=1). The absolute refractory period was measured using nonlinear regression of the refractory period from a single phase exponential decay of stimulation amplitude normalized to threshold in MATLAB (MathWorks, Natick, MA, USA). Prior to fitting, screen were conducted to identify robust outliers within each amplitude group; see Statistics section for details.

Suppressing propagation experimental design. The effect of electrical stimulation on MC propagation velocity was measured in the isolated mouse colon. The investigator was not blinded to treatment group sham stimulation or electrical stimulation, and the same investigator performed data analyses. The actual and apparent velocity of the contraction wavefront was measured in sham stimulation and with electrical stimulation delivered in the proximal, middle, and distal colon. The actual velocity was calculated as the mean velocity of the contraction while it was propagating, before and after the temporary arrest induced by electrical stimulation. The apparent velocity was calculated as the net velocity for the continuous propagation from the oral to aboral end of the preparation.

Chemicals. Hexamethonium (no. H0879) and atropine (no. A0257) were obtained from Sigma-Aldrich (St. Louis, MO, USA). Both were prepared as stock solutions and kept refrigerated before being diluted to their appropriate concentrations before use: hexamethonium at 300 μM and atropine at 3 μM.

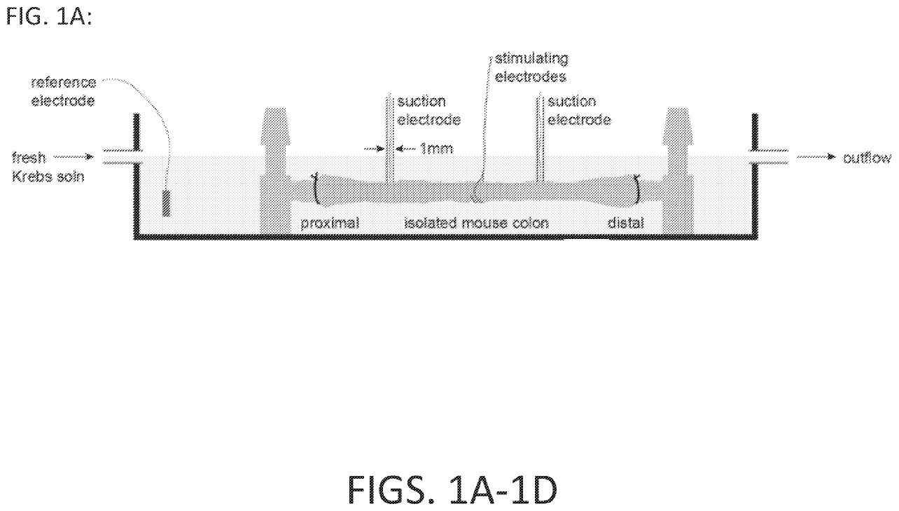

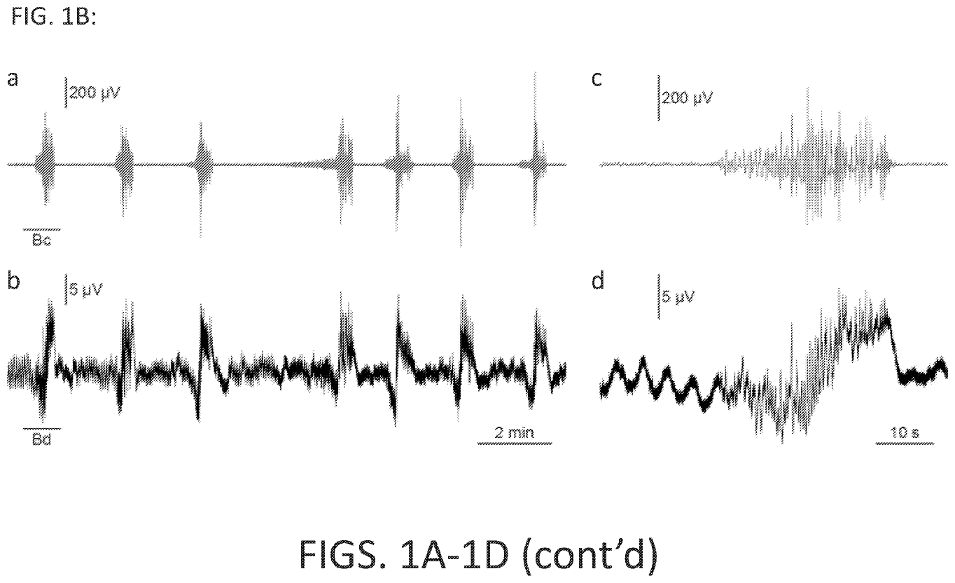

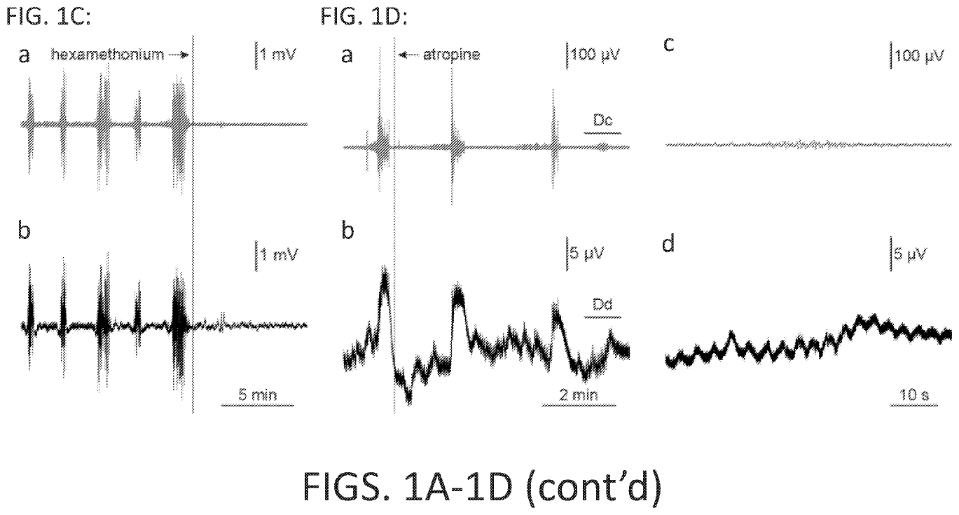

Myoelectric recordings. Myoelectric activity (EMG) in the isolated mouse colon was recorded from the serosal surface opposite of the mesenteric border using one or two suction electrodes (FIG. 1). DC-coupled extracellular recordings were used to detect slow waves, excitatory junction potentials (EJPs) and inhibitory junction potentials (IJPs). Experiments were conducted using two different experimental rigs with similar, but not identical equipment. Rig 1 recorded AC-coupled and DC-coupled EMG separately using ISO-80 (World Precision Instruments, Sarasota, FL, USA) and DAM-50 (World Precision Instruments) amplifiers, respectively. Both signals were processed with a HumBug 50 Hz low-pass filter (Quest Scientific, North Vancouver, BC, Canada). Rig 2 recorded DC-coupled EMG using a SR560 low noise amplifier (Stanford Research Systems, Sunnyvale, CA, USA) with 1 kHz low-pass filter and a 50 Hz digital low-pass filter. The DC-coupled recordings were transformed into AC-coupled recordings with digital high-pass filters at 0.5 Hz. Both rigs acquired data at 1 kHz sampling rate in LabChart 8 using PowerLab (AD Instruments, Colorado Springs, CO, USA).

Maintained physiological distension. Maintained physiological distension was used to evoke spontaneous, cyclic MCs. A metal rod inside silicone tubing was inserted through the lumen of each preparation. The diameter of distension was 2.6 mm and 2.1 mm at Rig 1 and Rig 2, respectively. The colon was stabilized by sutures holding either end over barbed tubing connectors (FIG. 1A).

Intraluminal Krebs perfusion. Intraluminal Krebs perfusion was used to evoke MCs by fluid distension. The colon was mounted on barbed tubing connectors and held in place with sutures. Warm Krebs solution was infused manually by syringe to distend the colon and evoke MCs.

Closed-loop controller. An online detector was used to control closed-loop electrical stimulation for measurement of the refractory period and for pacing of MCs during maintained physiological distension. The online detector used a first-order bandpass digital Butterworth filter between 1 and 5 Hz of data streamed from LabChart in MATLAB compared to a user-defined threshold to determine the state: an MC is occurring or an MC is not occurring. During state transitions, the online detector waited a 3 s interval to confirm the transition was robust before assigning the new state.

The closed-loop controller was written in MATLAB and interfaced with LabChart to deliver electrical stimulation. The closed-loop controller used two different functions to measure properties of the MC: binary search algorithm and entraining MCs. The binary search algorithm evaluated the ability of electrical stimulation to evoke an MC at varying delays after the preceding complex. An initial delay of 30 seconds was used to confirm that electrical stimulation and the online detector were working properly. The binary search algorithm was then allowed to identify the minimum delay necessary to evoke an MC under two conditions: following a spontaneous MC or following an evoked MC. Entraining MCs used the online detector to determine the end of an MC, and the closed-loop controller delivered electrical stimulation after a constant delay. The controller continued to deliver electrical stimulation after a constant delay following the determined end of a preceding MC until electrical stimulation failed to evoke an MC. For both the binary search algorithm and entrainment, electrical stimulation was defined to evoke an MC successfully if the onset of an MC was detected within 20 s of the beginning of electrical stimulation.

Electrical stimulation. Electrical stimulation was delivered as 100 pulses with 400 μs per phase at 20 pulses per second. Rig 1 used voltage-controlled stimulation (S48 and SIUSB, Grass Instruments) to deliver 50 V monophasic pulses via tungsten electrodes. Rig 2 used current-controlled stimulation to deliver symmetric, biphasic pulses at varying amplitudes via suction electrodes. At Rig 2, stimulating current was isolated (Model 2200, A-M Systems, Sequim, WA, USA) dc-filtered, and monitored across a 1 kΩ resistor. The threshold was coarsely determined in 0.1 mA increments as the minimum current necessary to evoke an MC.

Spatiotemporal diameter-mapping. In the preparation with intraluminal Krebs perfusion, a USB camera (C920 Webcam, Logitech, Newark, CA, USA) was used to capture colon diameter over time, as described previously (Barnes et al., 2014). In summary, the video was converted to a black-and-white silhouette of the colon and the diameter was approximated as a function of position in each recording. The diameter was converted to a grayscale value and represented on a map of colon position and time, with darker regions indicating larger diameter and lighter regions indicating smaller diameter. MATLAB was then used to calculate the differential of the diameter in time as an approximation of the location of the contraction wavefront.

Statistics. Summary values are reported as mean±standard deviation. The independent sample size, n, refers to the number of isolated mouse colons in a given experiment, also referred to as preparations. In the absence of prior statistical estimates, a small sample size was selected and the observed (post hoc) power was used to ensure the study was sufficiently powered. Wherever possible, statistical tests used paired analyses or included subject as a random effect. In cases in which repeated measurements were conducted under the same condition in the same preparation, the measurements are reported as the median value for the subject unless otherwise noted. Student's t-test and one-way ANOVA followed by Dunnett's test for multiple comparison were conducted in JMP Pro 14 (SAS, Cary, NC, USA). P-values and F-statistics (where appropriate) are reported for each statistical test. Outliers were defined as 4 spreads from the center using Huber M-Estimation.

### 5. EXAMPLES

Electrical stimulation of the enteric nervous system (ENS) is an attractive approach to modify gastrointestinal transit. Colonic motor complexes (CMCs) occur with a periodic rhythm, but the ability to elicit a premature CMC depends, at least in part, upon the intrinsic refractory properties of the ENS, which are presently unknown. The objectives of the present disclosure were to record myoelectric complexes (MCs, the electrical correlates of CMCs) in the smooth muscle and (i) determine the refractory periods of MCs, (ii) inform and evaluate closed-loop stimulation to repetitively evoke MCs, and (iii) identify stimulation methods to suppress MC propagation. The colon was dissected from male and female C57BL/6 mice, preserving the integrity of intrinsic circuitry while removing the extrinsic nerves, and measured properties of spontaneous and evoked MCs in vitro. Hexamethonium abolished spontaneous and evoked MCs, confirming the necessary involvement of the ENS for electrically-evoked MCs. Electrical stimulation reduced the mean interval between evoked and spontaneous CMCs (24.6±3.5 vs 70.6±15.7 s, p=0.0002, n=7). The absolute refractory period was 4.3 s (95% CI=2.8-5.7 s, R²=0.7315, n=8). Electrical stimulation lead to arrest of fluid distention-evoked propagating MC, and following cessation of stimulation propagation resumed at an increased velocity (n=9). The timing parameters of electrical stimulation increased the rate of evoked MCs, including the duration of entrained MCs, and provide insights into timing considerations for designing neuromodulation strategies to treat colonic dysmotility.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Cyclic MCs under maintained physiological distension. All isolated mouse colons with maintained physiological distension exhibited spontaneous cyclic MCs (n=14). Spontaneous cyclic neurogenic MCs occurred between quiescent periods of 85.7±26.8 s, and the mean duration of MCs was 25.0±5.5 s. DC-coupled recordings of MC activity often revealed myogenic slow waves (FIG. 1). Spontaneous cyclic MCs ceased following bath application of hexamethonium (300 μM, n=5, FIG. 1C), and subthreshold EJPs ceased after bath application of atropine (3 μM, n=3, FIG. 1D).

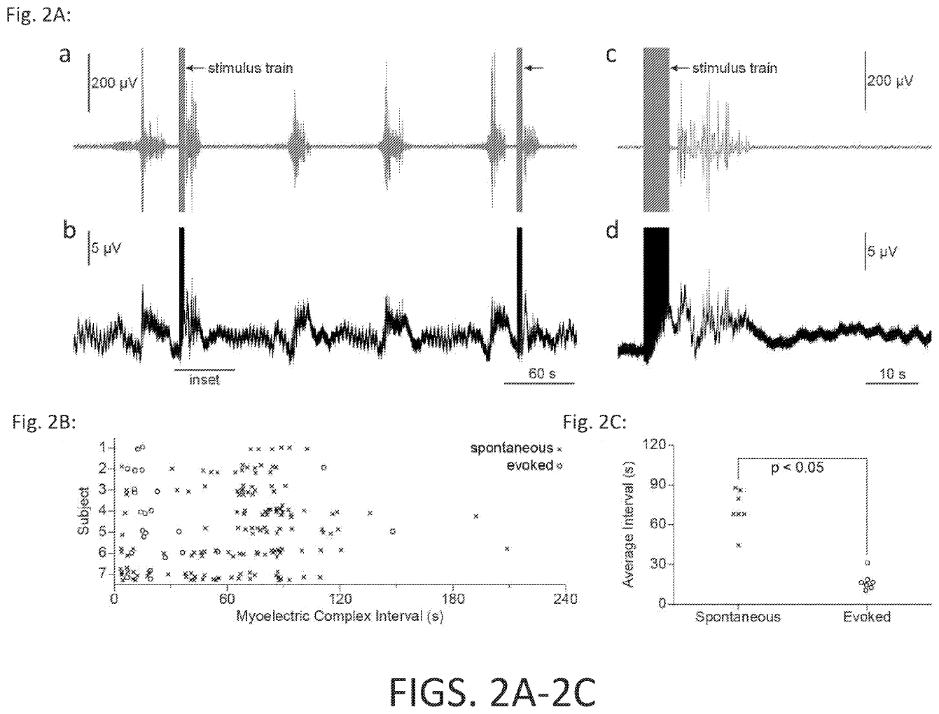

During cyclic spontaneous MCs, electrical stimulation at a location between the two recording electrodes evoked premature MCs in all preparations (FIG. 2). Electrical stimulation was delivered approximately 15 s after the prior MC (13.7±4.8 s, n=7). Evoked MCs were similar in duration to spontaneous MCs and also exhibited subthreshold EJPs. In 7 isolated mouse colons, the mean duration of evoked MCs was 22.0±5.6 s, and the mean duration of spontaneous MCs was 23.2±3.5 s. Electrical stimulation evoked premature cyclic MCs and reduced the interval between MCs to 24.6±13.0 s in comparison to 70.6±15.7 s in the absence of stimulation (p=0.0002, n=7). In two cases in different preparations, electrical stimulation was delivered too soon after the end of the preceding MC and did not appear to evoke an MC. This observation suggested that the MC had a refractory period or a minimum delay before a subsequent MC could be evoked.

### Example 2

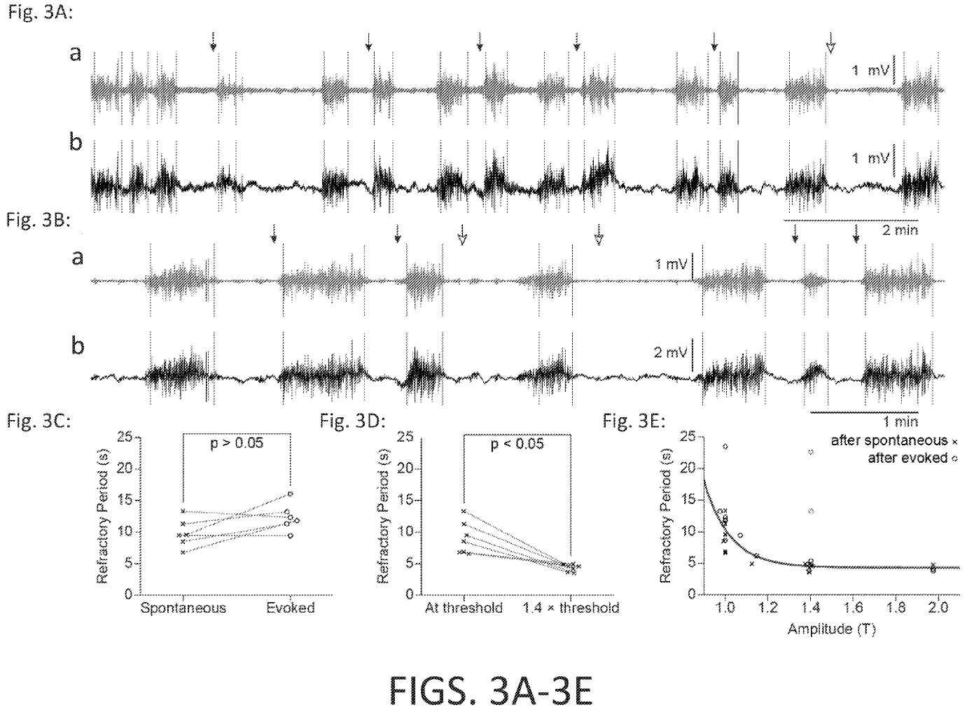

Refractory period of the MC. The refractory period was measured using a closed-loop controller. A stimulus train was delivered 30 s after the end of the preceding MC as a positive control. The stimulation threshold was approximated as the minimum current amplitude necessary to evoke an MC in each preparation, ranging between 0.2 and 1.7 mA. Then, a binary search algorithm was implemented to estimate the minimum delay necessary to evoke an MC after a spontaneous MC and after an evoked MC (FIGS. 3A-3B).

The refractory period at threshold after a spontaneous MC (9.9±2.3 s) was not different from the refractory period after an evoked MC (12.34±2.2 s, p=0.0850, n=6, FIG. 3C). Increasing stimulation amplitude to 140% of threshold decreased the refractory period after a spontaneous MC from 9.0 s±2.6 to 4.4±0.6 s (p=0.0042, n=7, FIG. 3D). The estimated absolute refractory period was 4.3 s (95% CI=3.0-5.6 s, FIG. 3E).

### Example 3

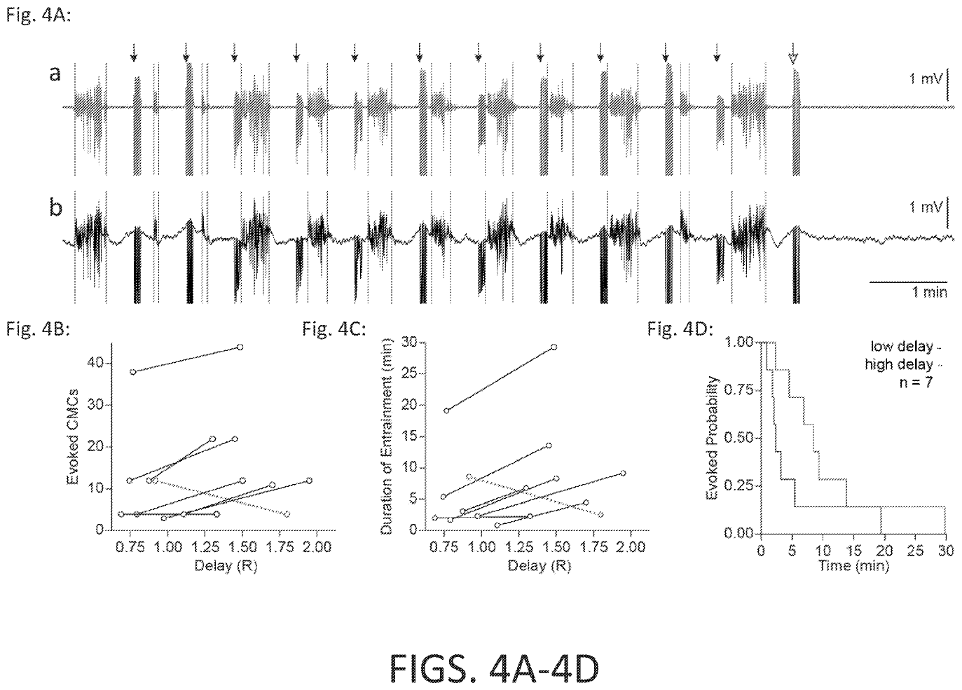

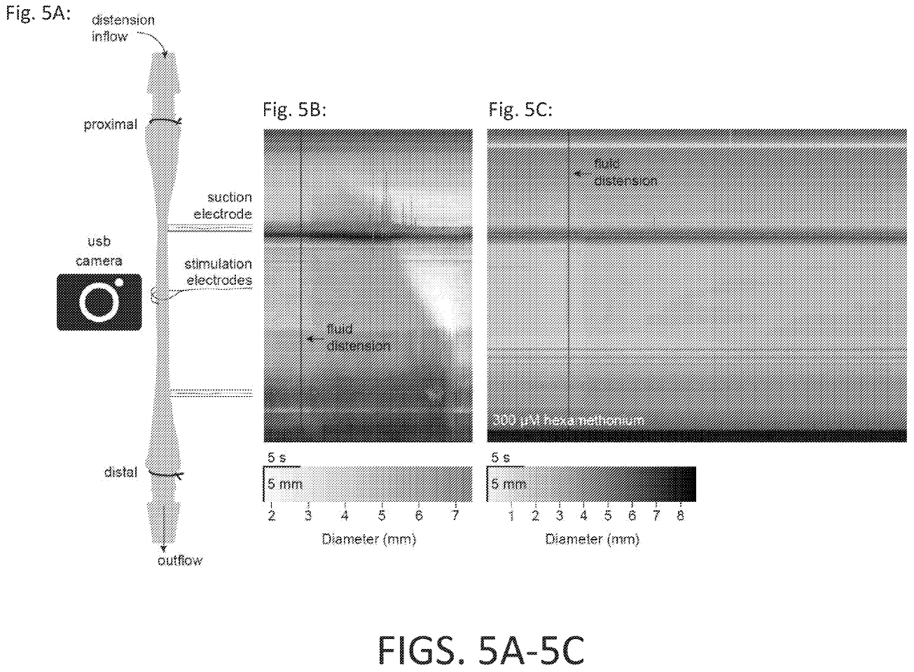

MC entrainment. An online detector was used to trigger closed-loop stimulation to evoke MCs with the intention of continually evoking entrained activity, i.e., pacing MCs. Stimulus trains were delivered at a constant delay after the previous MC until the stimulus train failed to evoke an MC (FIG. 4A). The number of successfully evoked MCs and the duration of entrainment were compared between two conditions: delay approximately equal to the refractory period (1R) or twice the refractory period (2R). Increasing the delay increased the number of evoked MCs and the duration of entrainment in 6 out of 8 preparations (FIGS. 4B-4C). One preparation was excluded from analyses because the ability to evoke MCs was not stable during the course of measurements. The number of evoked MCs at 1R and 2R delay was 11.0±12.5 and 18.1±13.1, respectively (p=0.0016, n=7), and the duration of entrainment at 1R and 2R delay was 5.0±6.5 min and 10.7±9.2 min (p=0.0043, n=7), respectively. Doubling the delay during closed-loop stimulation increased the median duration of continuing to evoke MCs by 6.1 min or 360% (FIG. 4D).

### Example 4

MC propagation suppressed by electrical stimulation. In empty preparations, isolated colons were distended by intraluminal fluid injection, and MCs were detected from both proximal and distal electrodes. Approximating the relative diameter from spatiotemporal images revealed propagating contractions that correlated in time and position with MCs (FIG. Contractions originated in the proximal colon and propagated the entire length of the isolated colon. Following bath application of 300 μM hexamethonium, MCs were no longer evoked by fluid injection (n=6).

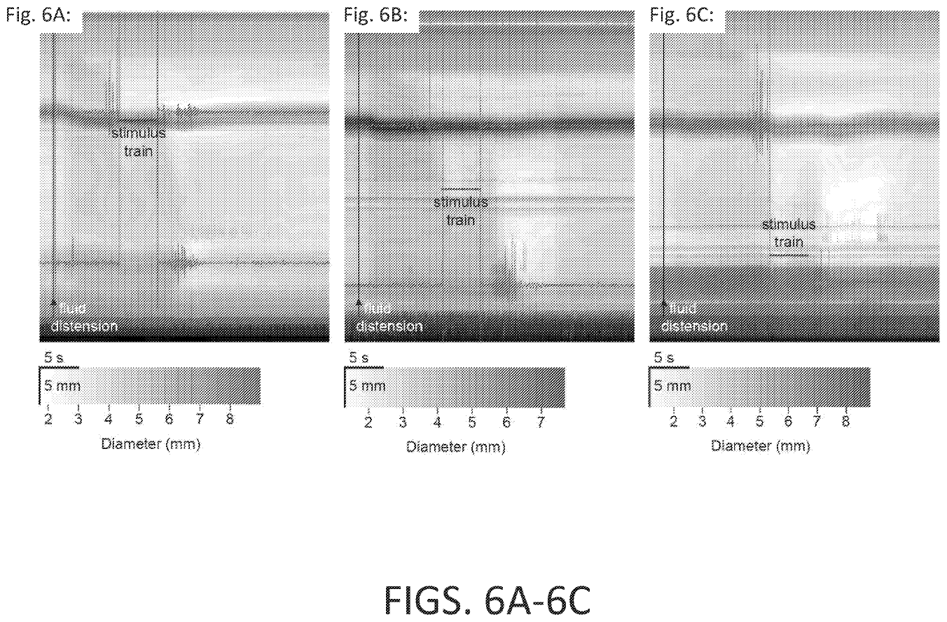

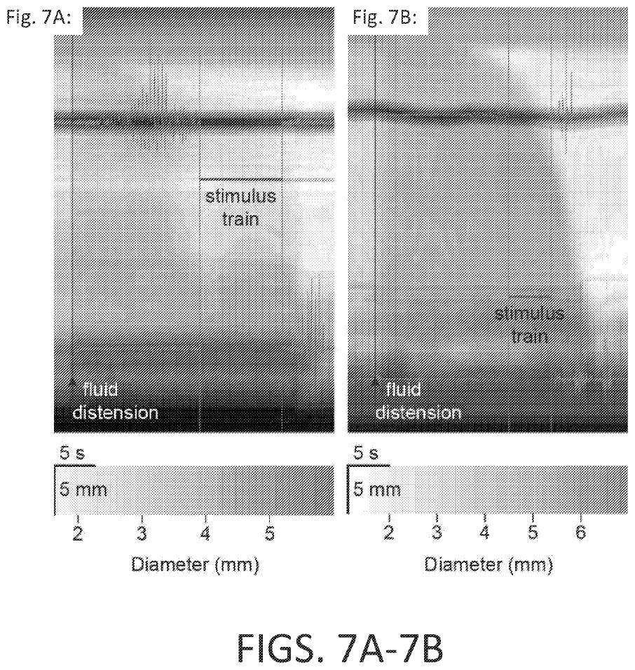

Electrical stimulation temporarily halted propagation of MCs evoked by fluid distension (FIG. 6). The temporary pause in propagation lasted 5 s, equivalent to the duration of stimulation. During the arrest of the contraction, intraluminal fluid transiently back flowed until the stimulus ceased. When the stimulus train was terminated, propagation of the MC continued from the location where it had halted. Temporary arrest of the contraction was reproducible at the proximal, middle, and distal colon. When the duration of stimulation was increased to 10 s, the contraction was arrested for the duration of stimulation (n=2, FIG. 7A). In instances in which the stimulation was delivered too early, i.e., the electrical stimulus ended before the MC arrived at the location of stimulation, then the MC propagated uninterrupted along the length of colon (n=4, FIG. 7B).

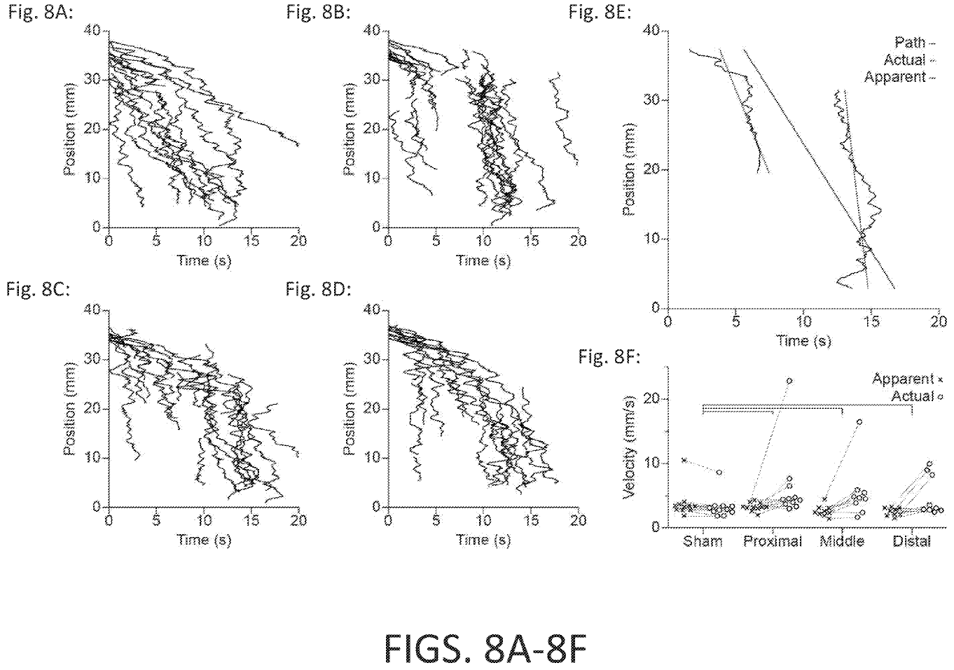

Tracking the position of the contractions in time revealed steady propagation in the unstimulated condition and discontinuous propagation with electrical stimulation (FIGS. 8A-8D). The contraction propagation without stimulation had a velocity of 3.3±1.9 mm/s. The discontinuous paths of propagation with electrical stimulation exhibited a clear arrest in propagation for the duration of the stimulation. However, the time for contractions to reach the distal colon was unchanged between unstimulated and stimulated propagating contractions. Subsequent analysis demonstrated that the contraction propagation velocity increased after the pause caused by electrical stimulation.

The actual velocity and apparent velocity of the contraction propagation were estimated (FIG. 8E). In the absence of stimulation, the mean actual (3.3±1.9 mm/s) and apparent (3.8±2.3 mm/s) velocities were within 0.5 mm/s (n=11). In cases in which electrical stimulation temporarily arrested propagation, the actual velocity was greater than the apparent velocity (p=0.00002, n>9, FIG. 8F): the mean actual velocity and mean apparent velocity for proximal stimulation were 6.3±5.7 mm/s and 3.3±0.7 mm/s, for middle stimulation were ±4.3 mm/s and 2.7±0.9 mm/s, and for distal stimulation were 5.0±3.1 mm/s and 2.5±mm/s, respectively.

1. A method of treating gastrointestinal dysmotility in a subject, the method comprising:
applying at least one temporal pattern of electrical stimulation to a target nerve or a set of target nerves in a subject having at least one symptom of a gastrointestinal hypermotility disorder and/or a hypomotility disorder;
wherein the application of the at least one temporal pattern of electrical stimulation prior to a refractory period suppresses contractions and motility, thereby treating the hypermotility disorder; and/or
wherein the application of the at least one temporal pattern of electrical stimulation after a refractory period stimulates contractions and motility, thereby treating the hypomotility disorder.

2. The method according to claim 1, further comprising selecting the at least one temporal pattern of electrical stimulation based on the subject having one or more symptoms of gastrointestinal hypermotility and/or hypomotility.

3. The method according to claim 1, wherein the at least one temporal pattern of electrical stimulation applied prior to the refractory period comprises a continuous pattern of electrical stimulation.

4. The method according to claim 1, wherein:
the at least one temporal pattern of electrical stimulation applied prior to the refractory period comprises a burst pattern of electrical stimulation having an interburst interval less than or equal to the refractory period; or
the at least one temporal pattern of electrical stimulation applied after the refractory period comprises a burst pattern of electrical stimulation.

5. (canceled)

6. The method according to claim 1, wherein the refractory period is determined based on the time between spontaneous gastrointestinal contractions.

7. The method according to claim 1, wherein the target nerve or set of target nerves comprise an extrinsic nerve or set of extrinsic nerves, or intrinsic (enteric) nerves.

8-9. (canceled)

10. The method according to claim 7, wherein the extrinsic nerve or set of extrinsic nerves, or the intrinsic (enteric) nerves innervate the gastrointestinal tract.

11. The method according to claim 1, wherein the refractory period is determined based on the time between contractions evoked by applied electrical stimulation of extrinsic nerves or intrinsic nerves.

12. The method according to claim 1, wherein the refractory period ranges from about 10 seconds to about 60 seconds, and wherein the subject is a human.

13. The method according to claim 3, wherein the continuous pattern of electrical stimulation comprises pulses delivered at a constant frequency for a pre-determined length of time.

14. The method according to claim 13, wherein the frequency is from about 1 Hz to about 50 Hz, and/or wherein the pre-determined length of time is from about 1 second to about 60 seconds.

15. (canceled)

16. The method according to claim 4, wherein the burst pattern of electrical stimulation comprises an interburst interval that is greater than the refractory period.

17. The method according to claim 4, wherein the burst pattern of electrical stimulation comprises bi-phasic pulses, and/or wherein each phase of the pulses within the burst pattern of electrical stimulation is from about 50 μs to about 1000 μs.

18. (canceled)

19. The method according to claim 4, wherein the burst pattern of electrical stimulation comprises about 50 to about 150 pulses per burst, and/or an intraburst pulse repetition frequency from about 1 Hz to about 50 Hz.

20-21. (canceled)

22. The method according to claim 1, wherein the subject is a human.

23. The method according to claim 1, wherein the at least one symptom of gastrointestinal hypermotility comprises early satiety, nausea, vomiting, bloating, diarrhea, constipation and/or involuntary weight loss.

24. The method according to claim 1, wherein the at least one symptom of gastrointestinal hypomotility comprises nausea, vomiting, abdominal pain, abdominal swelling (distention) and/or constipation.

25-27. (canceled)

28. A method of treating gastrointestinal dysmotility in a subject, the method comprising:
programming a pulse generator to output at least one temporal pattern of electrical stimulation to a target nerve or set of target nerves in a subject having at least one symptom of a gastrointestinal hypermotility disorder and/or a hypomotility disorder; and
delivering the at least one temporal pattern of electrical stimulation to the subject prior to a refractory period to suppress contractions and motility, thereby treating the hypermotility disorder; and/or
delivering the at least one temporal pattern of electrical stimulation to the subject after a refractory period to stimulate contractions and motility, thereby treating the hypomotility disorder.

29. The method according to claim 28, wherein:
the at least one temporal pattern of electrical stimulation applied prior to the refractory period comprises a continuous pattern of electrical stimulation;
the at least one temporal pattern of electrical stimulation applied prior to the refractory period comprises a burst pattern of electrical stimulation having an interburst interval less than or equal to the refractory period; or
the at least one temporal pattern of electrical stimulation applied after the refractory period comprises a burst pattern of electrical stimulation having an interburst interval greater than the refractory period.

30-31. (canceled)

32. The method according to claim 28, wherein the at least one temporal pattern of electrical stimulation is delivered to a single subject at one or more time points.
